(19) United States
(12) Patent Application Publication
(10) Pub. No.: US 2019/0365442 A1
(43) Pub. Date: Dec. 5, 2019

(54) BONE SCREWS WITH ENHANCED FATIGUE RESISTANCE AND RELATED KITS AND METHODS

(71) Applicant: ARTHROSURFACE, INC., Franklin, MA (US)

(72) Inventors: Nikhil T. JAWRANI, Framingham, MA (US); Steven W. EK, Bolton, MA (US)

(21) Appl. No.: 16/426,364

(22) Filed: May 30, 2019

Related U.S. Application Data
(60) Provisional application No. 62/679,239, filed on Jun. 1, 2018.

(51) Int. Cl. A61B 17/86; A61B 17/80

(52) U.S. Cl. CPC A61B 17/8605; A61B 17/7059; A61B 17/8625; A61B 17/8033

(57) ABSTRACT

The present bone screws—and kits and methods utilizing such bone screws—provide enhanced fatigue resistance (EFR), increased fixation strength, and/or improved tactile feedback. The enhanced fatigue resistance can increase the working life of a bone screw and reduce the possibility of a bone screw cracking or breaking and possible adverse implications thereof. The improved tactile feedback can improve usability by maintaining a level friction and resistance (during insertion of the screws into bone) that is familiar to health care providers. The present bone screws, kits, and methods may be configured and/or implemented for use in the repair of cancellous and/or cortical bone.

### CROSS-REFERENCE TO RELATED APPLICATION

The present application claims priority to U.S. Provisional Patent Application No. 62/679,239 filed Jun. 1, 2018, the disclosure of which application is hereby incorporated by reference in its entirety.

### TECHNICAL FIELD

The present application relates generally to orthopedic treatments, and more particularly, but not by way of limitation, to devices, apparatuses, kits, and methods for modifying bone (e.g., for stabilizing or repairing fractured bone).

### BACKGROUND

Bone screws have been used to treat bone disorders or injuries for decades. Treatment typically involves stabilizing the bone and providing compression in order to facilitate the healing process. A typical bone screw is cylindrical in shape with helical threads along at least a distal portion of its length. The threads, when rotated into the bone, can convert torsional forces into compression forces to bring together two objects (e.g., pieces of a fractured bone). Examples of bone screw use in orthopedics include, for example, attachment of implants to bone, or bone to bone fixation, or soft tissue fixation to bone.

Despite advances in bone screw design, the basic components have largely remained the same. The main components of the typical bone screw are the head, the shaft, and the tip. The head serves as a buttress to prevent the screw from sinking into the bone, as well as providing a point of engagement for a screwdriver to rotate the screw into position. Screwdriver engagement varies, but modern bone screws often employ a hexagonal recess. The shaft of the bone screw has a core diameter, shaft diameter, and thread diameter. The shaft diameter is the diameter of the shaft where there is no thread. The core diameter is the smallest diameter of the threaded section of the shaft. The thread diameter is the diameter of the widest part of the threaded section. While some bone screws have a threaded diameter that is larger than the shaft diameter, other bone screws may have a thread diameter that is the same as the shaft diameter. The ultimate strength of the bone screw itself, however, may be limited by the smallest one of the shaft diameter and the core diameter.

Bone screws may include any of various biocompatible materials, for example, stainless steel, titanium, and/or any of various alloys known in the art.

### SUMMARY

The present bone screws—and kits and methods utilizing such bone screws—provide enhanced fatigue resistance (EFR), increased fixation strength, and/or improved tactile feedback. The enhanced fatigue resistance can increase the working life of a bone screw and reduce the possibility of a bone screw cracking or breaking and possible adverse implications thereof. The improved tactile feedback can improve usability by maintaining a level friction and resistance (during insertion of the screws into bone) that is familiar to health care providers. The present bone screws, kits, and methods may be configured and/or implemented for use in the repair of cancellous and/or cortical bone.

In some configurations, the present bone screws comprise: an elongated shank extending from a proximal end to a distal tip (e.g., a rounded distal tip), the shank defining one or more (e.g., helical) threads along at least a portion of a length of the shank; a head coupled to the proximal end of the shank, the head having a transverse dimension that is larger than a corresponding transverse dimension of the proximal end of the shank; and a titanium Type II anodization (per AMS 2488) on the shank. In some configurations, the Type II anodization comprises titanium and/or its alloys. The Type II anodized surface layer may be roughened over at least part of the thread(s), for example by abrasive blasting (e.g., glass bead blasting, aluminum oxide (Al₂O₃) blasting, etc.) or etching. The Type II anodized surface layer may also extend over the head. The head may be unitary with the shank, and/or may define a recess configured to receive a driver. The shank and head may each comprise at least one material such as titanium and/or its alloys (e.g., Ti-6Al-4V Type II anodized per AMS 2488), or other materials on which similar, structural surface finish layers may be produced. A distal side of each thread may be concave, and/or a proximal side of each thread may be planar or convex.

In some configurations of the present bone screws, the threads may be configured as self-tapping and/or self-drilling thread(s). For example, the shank may define a longitudinal, self-tapping notch extending through a portion of the thread(s) from the distal tip toward the proximal head.

In some configurations of the present bone screws, the shank may define a longitudinal channel extending through the distal tip toward the proximal head and/or through the proximal head.

In some configurations of the present bone screws, part of the shank is not threaded.

In some configurations of the present bone screws, the head may be unitary with a single use driver that is configured to be broken or otherwise separated from the head after the screw is inserted into bone.

In another aspect of the present disclosure, a method of manufacturing a bone screw is disclosed. Some implementations of the present method for manufacturing a bone screw include providing a Type II anodized surface layer overlying at least part of one or more thread(s) defined by an elongated shank of a bone screw, the shank extending from a proximal end to a distal tip (e.g., a rounded distal tip), where the shank defines the (e.g., helical) thread(s) along at least a portion of a length of the shank, the bone screw further comprising a head coupled to the proximal end of the shank, the head having a transverse dimension that is larger than a corresponding transverse dimension of the proximal end of the shank. Some implementations of the present methods additionally or alternatively include roughening a Type II anodized surface layer that overlies at least part of one or more thread(s) defined by an elongated shank of a bone screw, the shank extending from a proximal end to a distal tip (e.g., a rounded distal tip), where the shank defines the (e.g., helical) thread(s) along at least a portion of a length of the shank, the bone screw further comprising a head coupled to the proximal end of the shank, the head having a transverse dimension that is larger than a corresponding transverse dimension of the proximal end of the shank. Roughening may comprise abrasive blasting (e.g., glass bead blasting, such as aluminum oxide (Al₂O₃) blasting, etc.) or etching. The distal side of each thread may be concave, and the proximal side of each thread may be planar or convex. The head may be unitary with the shank, and/or may define a recess configured to receive a driver. The shank and head may each comprise at least one material such as titanium and/or its alloys (e.g., Ti-6Al-4V Type II anodized per AMS 2488), or other materials on which similar, structural surface finish layers may be produced.

The threads may be configured as self-tapping and/or self-drilling thread(s), where the distal side of each thread may be concave, and the proximal side of each thread may be planar or convex. For example, the shank may define a longitudinal, self-tapping notch extending through a portion of the thread(s) from the distal tip toward the proximal head. In some implementations, the shank may define a longitudinal channel extending through the distal tip toward the proximal head and/or through the proximal head. In some implementations, part of the shank is not threaded.

In some implementations, the head is unitary with a single-use driver that is configured to be broken or otherwise separated from the head after the screw is inserted into bone.

In another aspect of the present disclosure, a kit including one or more of the bone screw configurations disclosed in this application, and a package within which the one or more bone screws is sealed, is disclosed. In such configurations, one or more of the elements of the kit are sterile.

In another aspect of the present disclosure, a method of modifying a bone is disclosed. Some implementations of the present method of modifying bone include providing a bone screw of any one of the present bone screw configurations, and rotating the bone screw into the bone to engage the threads with the bone.

The term “roughened” describes the texture of the anodized surface layer of the bone screw, which can be formed, for example, by bead blasting to create local compressions of the surface layer and/or remove localized portions of the anodized surface layer. With Type II anodized surface layers, such bead blasting will typically not remove the full thickness of the anodized surface layer.

The term “coupled” is defined as connected, although not necessarily directly, and not necessarily mechanically; two items that are “coupled” may be unitary with each other. The terms “a” and “an” are defined as one or more unless this disclosure explicitly requires otherwise. The term “substantially” is defined as largely but not necessarily wholly what is specified (and includes what is specified; e.g., substantially 90 degrees includes 90 degrees and substantially parallel includes parallel), as understood by a person of ordinary skill in the art. In any disclosed configuration, the term “substantially” may be substituted with “within [a percentage] of” what is specified, where the percentage includes 0.1, 1, 5, and/or 10 percent.

The terms “comprise” (and any form of comprise, such as “comprises” and “comprising”), “have” (and any form of have, such as “has” and “having”), “include” (and any form of include, such as “includes” and “including”) and “contain” (and any form of contain, such as “contains” and “containing”) are open-ended linking verbs. As a result, an apparatus or kit that “comprises,” “has,” “includes” or “contains” one or more elements possesses those one or more elements, but is not limited to possessing only those elements. Likewise, a method that “comprises,” “has,” “includes” or “contains” one or more steps possesses those one or more steps, but is not limited to possessing only those one or more steps.

Further, a device, apparatus, kit, or method that is configured in a certain way is configured in at least that way, but it can also be configured in other ways than those specifically described.

The terms “comprise” (and any form of comprise, such as “comprises” and “comprising”), “have” (and any form of have, such as “has” and “having”), and “include” (and any form of include, such as “includes” and “including”) are open-ended linking verbs. As a result, an apparatus that “comprises,” “has,” or “includes” one or more elements possesses those one or more elements, but is not limited to possessing only those elements. Likewise, a method that “comprises,” “has,” or “includes” one or more steps possesses those one or more steps, but is not limited to possessing only those one or more steps.

Any configuration of any of the present devices, apparatuses, kits, and methods can consist of or consist essentially of—rather than comprise/include/contain/have—any of the described steps, elements, and/or features. Thus, in any of the claims, the term “consisting of” or “consisting essentially of” can be substituted for any of the open-ended linking verbs recited above, in order to change the scope of a given claim from what it would otherwise be using the open-ended linking verb.

The feature or features of one configuration may be applied to other configurations, even though not described or illustrated, unless expressly prohibited by this disclosure or the nature of the configurations.

Some details associated with the configurations are described above and others are described below.

### BRIEF DESCRIPTION OF THE DRAWINGS

The following drawings illustrate by way of example and not limitation. For the sake of brevity and clarity, every feature of a given structure is not always labeled in every figure in which that structure appears. Identical reference numbers do not necessarily indicate an identical structure. Rather, the same reference number may be used to indicate a similar feature or a feature with similar functionality, as may non-identical reference numbers. The figures are drawn to scale for at least the configurations shown.

FIG. 1A shows a perspective view of a first example of the present bone screws.

FIG. 1B shows a side view of the bone screw in FIG. 1A.

FIG. 2 shows a side view of a second example of the present bone screws.

FIG. 3 shows a side view of a third example of the present bone screws being inserted into two opposing ends of fractured bone.

FIG. 4 shows a side view of a fourth example of the present bone screws.

FIG. 5 shows a top view of a kit containing different sizes of the present bone screws.

FIG. 6 illustrates a process for roughening a Type II anodized surface layer of a bone screw.

FIG. 7A is a graph of results of cantilever-bending fatigue testing of Ti-6Al-4V Type II anodized (per AMS 2488) bone screws without any roughening/blasting steps (Group A, n=5), aluminum oxide (Al₂O₃) blasted bone screws without any anodization steps (Group B, n=5), and combination of Ti-6Al-4V Type II anodized (per AMS 2488)/Al₂O₃ blasted bone screws (Group C, n=5).

FIG. 7B is a graph of results of cantilever bending fatigue testing of Ti-6Al-4V Type II anodized (per AMS 2488) bone screws without any roughening/blasting steps (Group A, n=8), aluminum oxide (Al₂O₃) blasted bone screws without any anodization steps (Group B, n=8), and combination of Ti-6Al-4V Type II anodized (per AMS 2488)/Al₂O₃ blasted bone screws (Group C, n=8).

FIG. 8 is a graph of results of insertion-torque testing of Ti-6Al-4V Type II anodized (per AMS 2488) bone screws without any roughening/blasting steps (Group A), aluminum oxide (Al₂O₃) blasted bone screws without any anodization steps (Group B), and combination of Ti-6Al-4V Type II anodized (per AMS 2488)/Al₂O₃ blasted bone screws (Group C).

### DETAILED DESCRIPTION OF ILLUSTRATIVE CONFIGURATIONS

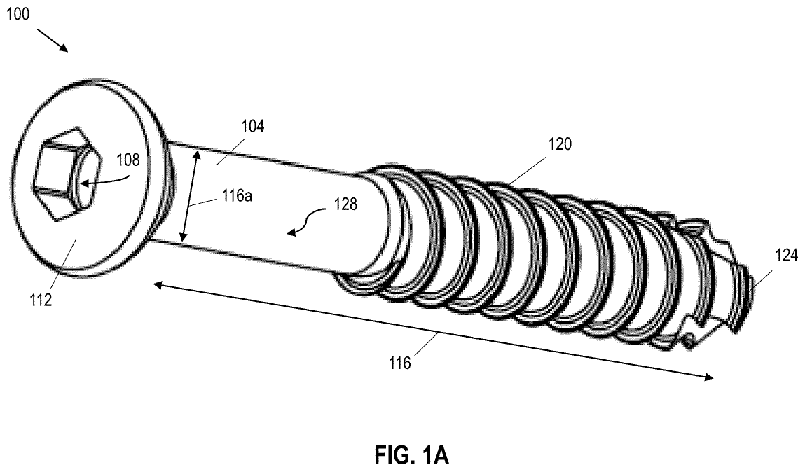

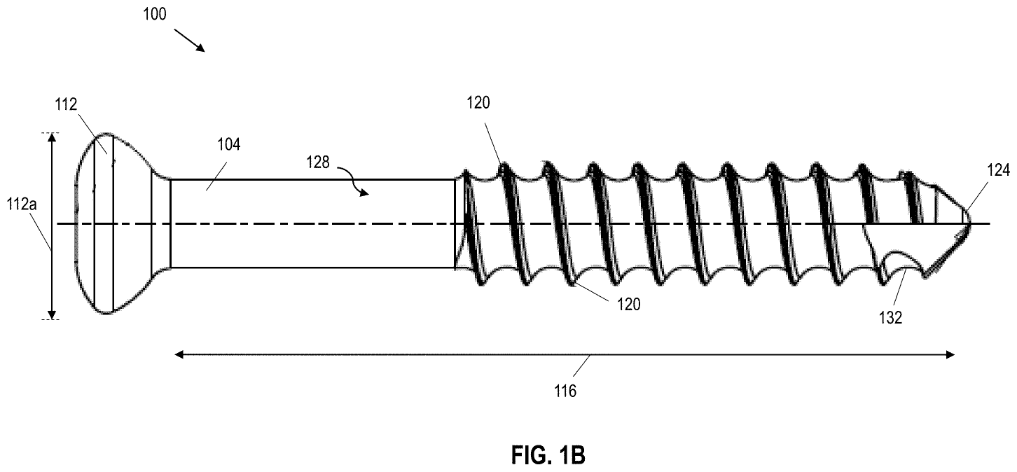

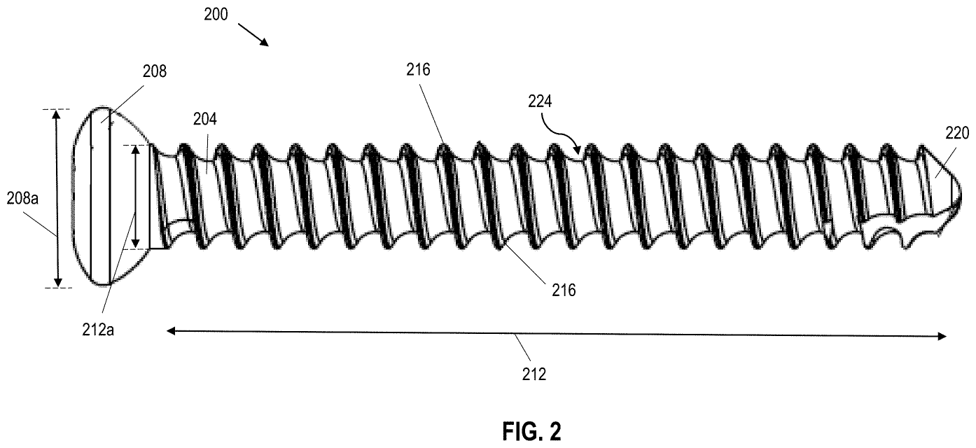

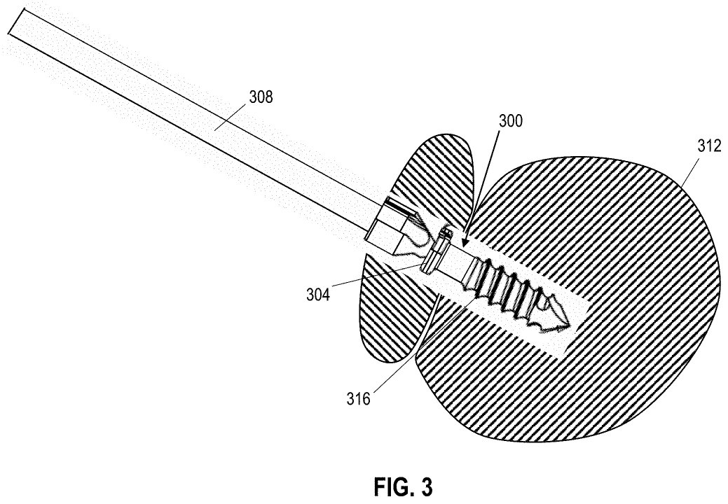

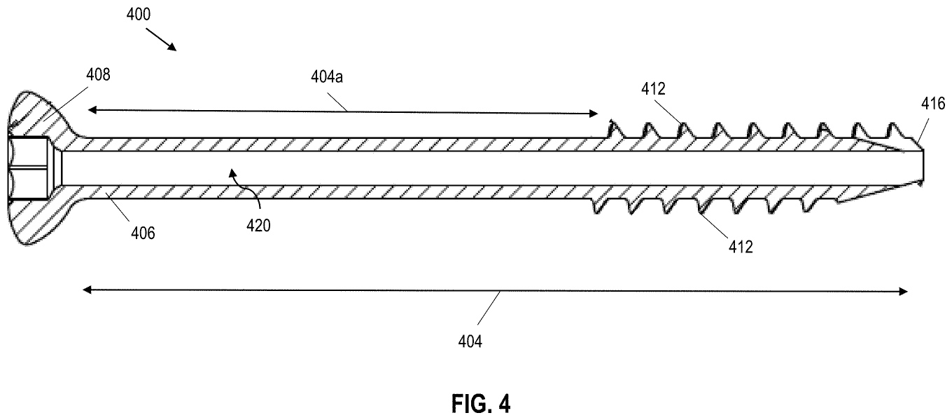

Referring now to the drawings, and more particularly to FIG. 1A-FIG. 6, FIG. 1A shows a perspective view of a first configuration of the present bone screw; FIG. 1B shows a side view of the bone screw in FIG. 1A; FIG. 2 shows a side view of a second configuration of the present bone screw; FIG. 3 shows a side view of a third configuration of the present bone screw being inserted into two opposing ends of fractured bone; FIG. 4 shows a cross-sectional side view of a fourth configuration of the present bone screw taken along a plane passing through the longitudinal channel.

In some configurations, such as the one shown in FIGS. 1A-1B, bone screw 100 comprises an elongated shank 116 extending from a proximal end 104 to a distal tip 124, with one or more threads 120 defined along at least a portion of a length of shank 116. As shown, a distal side (facing toward tip 124) of each thread may be concave, and a proximal side (facing away from tip 124) of each thread may be planar or convex. In this configuration, screw 100 also includes a head 112 coupled to proximal end 104 of shank 116. As shown, head 112 typically has a transverse dimension 112a that is larger than a corresponding transverse dimension 116a of proximal end 104 of shank 116. Screw 100 also has a Type II anodized surface layer 128 on shank 116 that provides an oxide layer that both (a) reduces surface imperfections and irregularities that might otherwise act as fracture-initiation points, and (b) forms a relatively-harder Ti-6Al-4V Type II anodized (per AMS 2488) surface layer that improves structural rigidity and resistance to bending of the screw. A Type II anodization can typically create oxide layer(s) that range from 1.8 μm to 25 μm in thickness. The Type II anodization can be applied titanium and/or its alloys (e.g., Ti-6Al-4V Type II anodized per AMS 2488), or other materials on which similar, structural surface finish layers may be produced.

In some configurations, the anodized surface layer 128 is roughened over at least part (e.g., all) of thread(s) 120. In some configurations, the roughened portion of the Type II anodized surface layer can be roughened by abrasive blasting, for example, aluminum oxide (Al₂O₃) blasting.

As best illustrated in FIG. 1B, in some configurations head 112 can be unitary with shank 116. In some configurations, thread(s) 120 can be configured as self-tapping and/or self-drilling threads. In some configurations, shank 116 can define a longitudinal, self-tapping notch 132 extending through a portion of thread(s) 120 from distal tip 124 toward proximal head 112. In some configurations, such as the one shown, part of shank 116 is not threaded. In some configurations, distal tip 124 is rounded. In some configurations, the thread(s) 120 are helical. In some configurations, shank 116 and head 112 each comprise at least one material selected from the group of materials consisting of: a biocompatible metal, stainless steel, 316L stainless steel, and titanium (e.g., Ti-6Al-4V anodized type II per AMS 2488).

FIG. 2 depicts a second example 200 of the present bones screws. Screw 200 is substantially similar to screw 100, with the exception that bone screw 200 has thread(s) 216 running the entire length of shank 212.

FIG. 3 depicts a third example 300 of the present bone screws. Screw 300 is substantially similar to screw 200, with the exception that head 304 of screw 300 is unitary with single use driver 308 that is configured to be broken or otherwise separated from head 304 after bone screw 300 is inserted into bone 312. As shown, in a surgical procedure to repair fractured bone, the two opposing ends of fractured bone 312 are modified by providing bone screw 300 and rotating bone screw 300 via single use driver 308 into bone 312 to engage threads 316 with bone 312. The Type II anodized surface layer of bone screw 300, which has been roughened over at least part of the threads, provides enhanced fatigue resistance, increased fixation strength, and/or improved tactile feedback when rotating bone screw 300 via single use driver 308 into position. The enhanced fatigue strength increases the working life of a bone screw and reducing the possibility of a bone screw cracking or breaking and possible adverse implications thereof. In some configurations, the Type II anodized surface layer also extends over the head. The improved tactile feedback can improve usability by maintaining a level friction and resistance (during insertion of the screws into bone) that is familiar to health care providers. Once screw 300 is in position, single use driver 308 is then broken or otherwise separated from head 304, leaving the remaining portion of bone screw 300 in place.

FIG. 4 depicts a fourth example 400 of the present bone screws. Screw 400 is substantially similar to screw 100, with the exception that the shank 404 of screw 400 defines a longitudinal channel 420 extending through distal tip 416 toward proximal head 408. In some configurations, such as the one shown, longitudinal channel 420 also extends through proximal head 408.

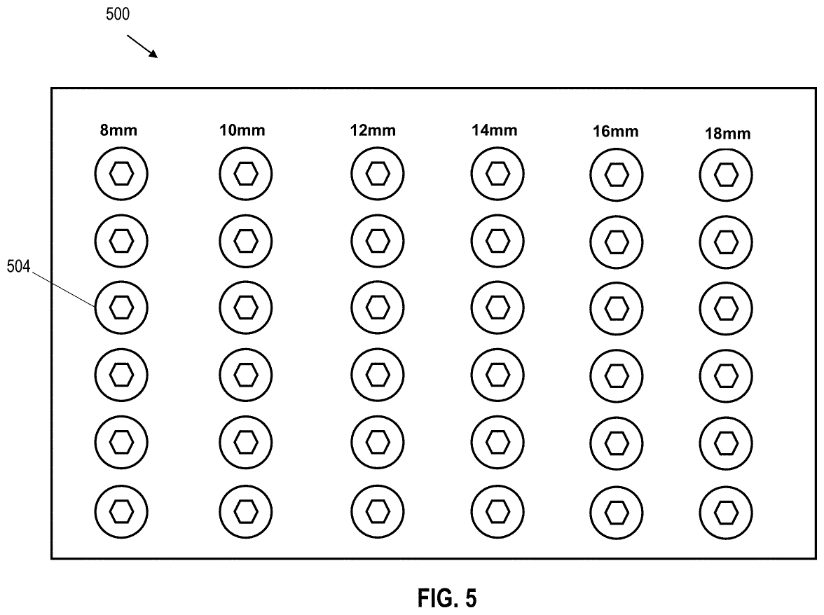

FIG. 5 shows a top view of a kit that includes one or more of the present bone screws in various sizes. As shown, kit 500 includes a plurality of bone screws 504 (e.g., 100, 200, 300, 400) of various sizes.

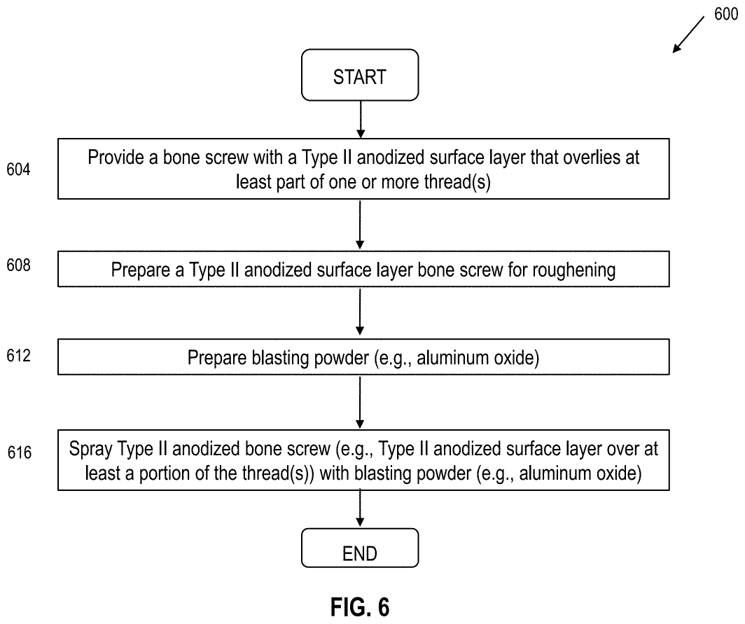

FIG. 6 shows a flow chart depicting a process for manufacturing at least some configurations of the present bone screws. Some implementations of the present method of manufacturing any of the bone screw configurations disclosed herein comprise: providing a bone screw with a Type II anodized surface layer overlying at least part of (e.g., all of) one or more thread(s) defined by an elongated shank of a bone screw, the shank extending from a proximal end to a distal tip (e.g., a rounded distal tip), where the shank defines the (e.g., helical) thread(s) along at least a portion of a length of the shank, the bone screw further comprising a head coupled to the proximal end of the shank, the head having a transverse dimension that is larger than a corresponding transverse dimension of the proximal end of the shank. In some configurations, the Type II anodized surface layer extends over the entire screw (i.e., including the head and the shank).

Some implementations of the present methods additionally or alternatively include roughening a Type II anodized surface that overlies at least part of (e.g., all of) one or more thread(s) defined by an elongated shank of a bone screw, the shank extending from a proximal end to a distal tip (e.g., a rounded distal tip), where the shank defines the (e.g., helical) thread(s) along at least a portion of a length of the shank, the bone screw further comprising a head coupled to the proximal end of the shank, the head having a transverse dimension that is larger than a corresponding transverse dimension of the proximal end of the shank. In some configurations, the Type II anodized surface layer extends over the entire screw (i.e., including the head and the shank). In some implementations of the present methods, only a portion of the anodized surface layer is roughened (e.g., a portion of the anodized surface layer overlying some or all of the thread(s)). Roughening may comprise abrasive blasting (e.g., bead blasting, such as aluminum oxide (Al₂O₃) blasting). In some implementations of the present methods, the abrasive blasting can entail full removal of the surface oxide layer and roughening of the surface of the underlying, non-oxidized metal. In other implementations, the abrasive blasting can entail partial removal of the Type II anodized surface and roughening of the top surface of the Type II anodized metal. In some implementations of the present methods, the method further comprises prior to roughening the Type II anodized surface, anodizing the shank to form the Type II anodized surface. The head may be unitary with the shank, and/or may define a recess configured to receive a driver. The shank and head may each comprise at least one material selected from the group of materials consisting of: a biocompatible metal, stainless steel, 316L stainless steel, and titanium (e.g., titanium type II anodized).

In some implementations of the present methods, the threads may be configured as self-tapping and/or self-drilling thread(s), where the distal side of each thread may be concave, and the proximal side of each thread may be planar or convex. For example, the shank may define a longitudinal, self-tapping notch extending through a portion of the thread(s) from the distal tip toward the proximal head. In some implementations, the shank may define a longitudinal channel extending through the distal tip toward the proximal head and/or through the proximal head. In some implementations, part of the shank is not threaded.

In some implementations, the head is unitary with a single-use driver that is configured to be broken or otherwise separated from the head after the screw is inserted into bone.

FIG. 6 depicts a flow chart showing a process 600 for manufacturing any of the roughened bone screw configurations (e.g., 100, 200, 300, 400). The steps of process 600 will be described with reference to a bone screw with a Type II anodized surface that overlies at least part of (e.g., all of) one or more thread(s) defined by an elongated shank of the bone screw, the shank extending from a proximal end to a distal tip, where the shank defines the thread(s) along at least a portion of a length of the shank, the bone screw further comprising a head coupled to the proximal end of the shank, the head having a transverse dimension that is larger than a corresponding transverse dimension of the proximal end of the shank.

At step 604, a bone screw with a Type II anodized surface as previously described is provided. The bone screw may be made from at least one material selected from the group of materials consisting of: a biocompatible metal, stainless steel, 316L stainless steel, and titanium (e.g., titanium type II anodized). In some configurations, the head of the bone screw may be unitary with the shank. In some configurations, the threads of the bone screw are configured as self-tapping and/or self-drilling thread(s), where the distal side of each thread may be concave, and the proximal side of each thread may be planar or convex. In some configurations, the shank of the bone screw defines a longitudinal, self-tapping notch extending through a portion of the thread(s) from the distal tip toward the proximal head. In some configurations, the shank defines a longitudinal channel extending through the distal tip toward the proximal head. In some configurations, the longitudinal channel also extends through the proximal head. In some configurations, part of the shank is not threaded. In some configurations, the distal tip of the bone screw is rounded. In some configurations, the head of the bone screw defines a recess configured to receive a driver. In some configurations, the head is unitary with a single use driver that is configured to be broken or otherwise separated from the head after the screw is inserted into bone. In some configurations, the thread(s) of the bone screw are helical, where the distal side of each thread may be concave, and the proximal side of each thread may be planar or convex.

At step 608, a configuration of the bone screw as previously described is prepared for blasting. Preparation can include, but is not limited to, cleaning and loading the Type II anodized bone screw into an automated blasting machine or similarly prepared for manual blasting.

At step 612, the blasting powder is prepared for abrasive blasting. In one configuration, the blasting powder used can be aluminum oxide with a particulate size between 17.5 microns to 25 microns. Other high performance materials may also be used as the blasting medium (e.g., silicon carbide, sodium bicarbonate, glass bead, crushed glass, plastic media, and walnut shell) depending on the need. At block 616, the Type II anodized bone screw can be sprayed with the blasting powder either manually or with an automated blasting machine.

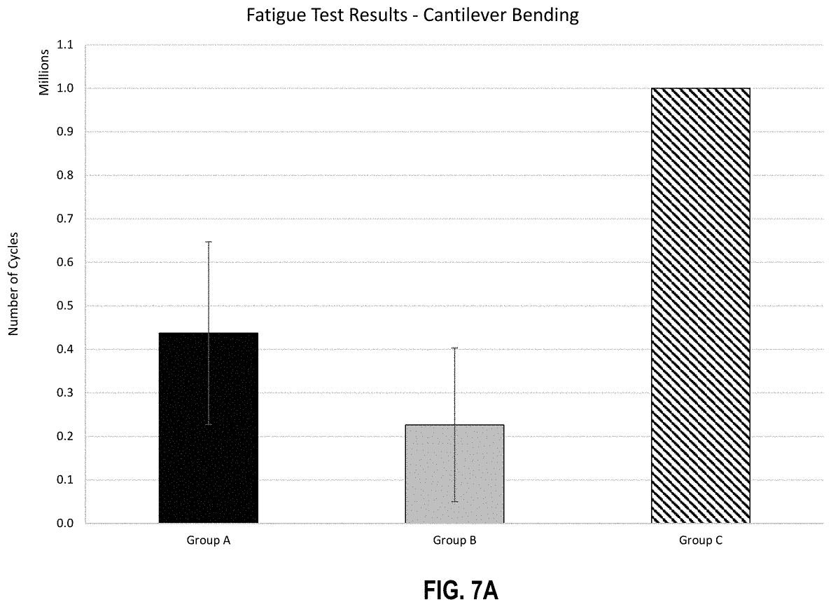

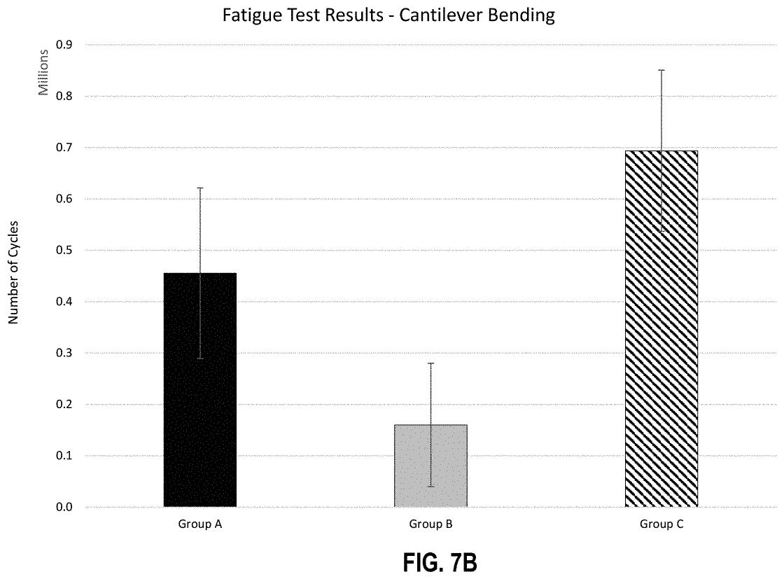

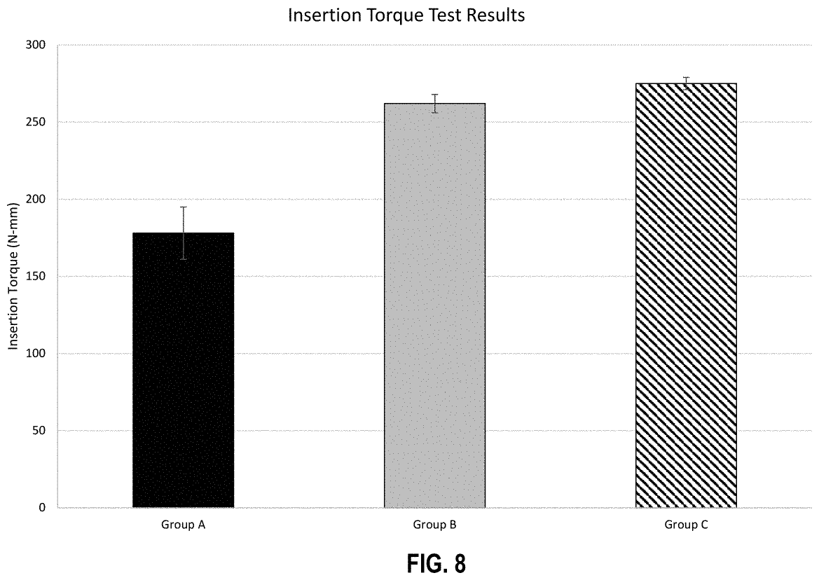

Referring now to FIGS. 7A-7B and 8, FIG. 7A shows the mean values of cycles to failure in fatigue testing of bone screws having a diameter of 4.0 mm, length of 32 mm and manufactured using Ti-6Al-4V titanium alloy per ASTM F136 under up to 1,000,000 cycles of cantilever bending at applied loads of 200 N and 225 N for: (A) bone screw samples that are entirely Type II anodized only (Group A); (B) bone screw samples that are entirely aluminum oxide (Al₂O₃) blasted only (Group B); and (C) bone screws that were Type II anodized entirely followed by Al₂O₃ blasting of the threads only (Group C). For example, when an applied force of at least 200N was applied to the bone screws in Groups A, B, and C (where n=5 for each Group), combining Type II anodized with Al₂O₃ blasting on the bone screws (Group C) more than doubled the fatigue life relative to the similar bone screws of Group A (Type II anodized only). In Group A (Type II anodized only), only 2 out of the 5 samples reached 1,000,000 cycles, whereas the other 3 samples fractured at or below about 10% of the full 1,000,000 cycle, indicating a wide variability in fatigue life of Type II anodized only bone screws. Similarly, combining Type II anodized with Al₂O₃ blasting on the bone screws (Group C) more than quadrupled the fatigue life relative to the similar bone screws of Group B (aluminum oxide (Al₂O₃) blasted only). In Group B (Al₂O₃ blasted only), only one out of five samples reached 1,000,000 cycles, whereas the other 4 samples fractured at or below about 5% of the full 1,000,000 cycle, again indicating a wide variability in fatigue life of Al₂O₃ blasted only bone screws.

Referring now to FIG. 7B as another example, when an applied force of at least 200N was applied to the bone screws in Groups A, B, and C (where n=8 for each Group), combining Type II anodized with Al₂O₃ blasting on the bone screws (Group C) increased the fatigue life more than 1.5 times relative to the similar bone screws of Group A (Type II anodized only). In Group A (Type II anodized only), only 3 out of the 8 samples reached 1,000,000 cycles, whereas the other 5 samples fractured at or below about 10% to 40% of the full 1,000,000 cycle, indicating a wide variability in fatigue life of Type II anodized only bone screws. Similarly, combining Type II anodized with Al₂O₃ blasting on the bone screws (Group C) more than quadrupled the fatigue life relative to the similar bone screws of Group B (aluminum oxide (Al₂O₃) blasted only). In Group B (Al₂O₃ blasted only), only one out of eight samples reached 1,000,000 cycles, whereas the other 7 samples fractured at or below about 8% of the full 1,000,000 cycle, again indicating a wide variability in fatigue life of Al₂O₃ blasted only bone screws. Thus, combining Type II anodizing with Al₂O₃ blasting of these bone screws provided enhanced fatigue resistance, increased fixation strength, and/or improved tactile feedback. The enhanced fatigue resistance can increase the working life of a bone screw and reduce the possibility of a bone screw cracking or breaking and possible adverse implications thereof.

FIG. 8 shows the results from insertion torque testing of the Group A, B, and C bone screw samples (where n=5 for each Group). As shown in the graph, combining Type II anodized with Al₂O₃ blasting of the threads only on the bone screws (Group C) increased insertion torque by about 35% relative to the similar bone screws of Group A (Type II anodized only). Similarly, combining Type II anodized with Al₂O₃ blasting on the bone screws (Group C) increased insertion torque about 5% relative to the similar bone screws of Group B (aluminum oxide (Al₂O₃) blasted only). These results show that combining Type II anodizing with Al₂O₃ blasting of the bone screws achieved a greater maximum insertion torque compared to similar bone screws that did not have a combination of a Type II anodized surface coat and Al₂O₃ blasted surface. The increase in insertion torque relative to anodized-only bone screws can give health care providers improved tactile feedback during insertion of the bone screws into bone.

The above specification and examples provide a complete description of the structure and use of exemplary configurations. Although certain configurations have been described above with a certain degree of particularity, or with reference to one or more individual configurations, those skilled in the art could make numerous alterations to the disclosed configurations without departing from the scope of this invention. As such, the various illustrative configurations of the present devices, apparatuses, kits, and methods are not intended to be limited to the particular forms disclosed. Rather, they include all modifications and alternatives falling within the scope of the claims, and configurations other than the one shown may include some or all of the features of the depicted configuration. For example, components may be combined as a unitary structure, and/or connections may be substituted. Further, where appropriate, aspects of any of the examples described above may be combined with aspects of any of the other examples described to form further examples having comparable or different properties and addressing the same or different problems. Similarly, it will be understood that the benefits and advantages described above may relate to one configuration or may relate to several configurations.

The claims are not intended to include, and should not be interpreted to include, means-plus- or step-plus-function limitations, unless such a limitation is explicitly recited in a given claim using the phrase(s) “means for” or “step for,” respectively.

1. A bone-screw, comprising:
an elongated shank extending from a proximal end to a distal tip, where the shank defines one or more threads along at least a portion of a length of the shank;
a head coupled to the proximal end of the shank, the head having a transverse dimension that is larger than a corresponding transverse dimension of the proximal end of the shank; and
a Type II (e.g., per AMS 2488 for titanium and its alloys) anodized surface layer on the shank.

2. The bone screw of claim 1, where the anodized surface layer extends over the head and/or is roughened over at least part of the thread(s).

3. The bone screw of claim 1, where the head is unitary with the shank and defines a recess configured to receive a driver, or the head is unitary with a single use driver that is configured to be broken or otherwise separated from the head after the screw is inserted into bone.

4. The bone screw of claim 1, where the threads are configured as self-tapping and/or self-drilling helical thread(s).

5. The bone screw of claim 4, where the shank defines a longitudinal, self-tapping notch extending through a portion of the helical thread(s) from the distal tip toward the proximal head.

6. The bone screw of claim 1, where the shank defines a longitudinal channel extending through the distal tip toward the proximal head or a longitudinal channel extending through the proximal head.

7. The bone screw of claim 1, where part of the shank is not threaded.

8. The bone screw of claim 1, where a portion of the anodized surface layer has been roughened by aluminum oxide blasting.

9. The bone screw of claim 1, where the distal tip is rounded.

10. The bone screw of claim 1, where the shank and head each comprise at least one material selected from the group of materials consisting of: a biocompatible metal, stainless steel, 316L stainless steel, and titanium.

11. A kit comprising:
a bone screw of claim 1; and
a package within which the bone screw is sealed.

12. A method of manufacturing a bone screw of claim 1, the method comprising:
forming a Type II anodized surface layer that overlies at least part of one or more thread(s) defined by an elongated shank of a bone screw, the shank extending from a proximal end to a distal tip, where the shank defines the thread(s) along at least a portion of a length of the shank, the bone screw further comprising a head coupled to the proximal end of the shank, the head having a transverse dimension that is larger than a corresponding transverse dimension of the proximal end of the shank.

13. A method of manufacturing a bone screw of claim 2, the method comprising:
roughening a Type II anodized surface layer that overlies at least part of one or more thread(s) defined by an elongated shank of a bone screw, the shank extending from a proximal end to a distal tip, where the shank defines the thread(s) along at least a portion of a length of the shank, the bone screw further comprising a head coupled to the proximal end of the shank, the head having a transverse dimension that is larger than a corresponding transverse dimension of the proximal end of the shank.

14. The method of claim 13, further comprising:
prior to roughening the Type II anodized surface layer, anodizing the shank to form the Type II anodized surface layer.

15. The method of claim 12, where the head is unitary with the shank and defines a recess configured to receive a driver, or the head is unitary with a single-use driver that is configured to be broken or otherwise separated from the head after the screw is inserted into bone.

16. The method of claim 12, where the threads are configured as self-tapping and/or self-drilling thread(s).

17. The method of claim 16, where the shank defines a longitudinal, self-tapping notch extending through a portion of the thread(s) from the distal tip toward the proximal head.

18. The method of claim 12, where the shank defines a longitudinal channel extending through the distal tip toward the head or a longitudinal channel extending through the proximal head.

19. The method of claim 12, where the shank and head each comprise at least one material selected from the group of materials consisting of: a biocompatible metal, stainless steel, 316L stainless steel, and titanium and its alloys.

20. A method of modifying a bone, comprising:
providing a bone screw of claim 1;
rotating the bone screw into the bone to engage the threads with the bone.

21. The method of claim 13, where the head is unitary with the shank and defines a recess configured to receive a driver, or the head is unitary with a single-use driver that is configured to be broken or otherwise separated from the head after the screw is inserted into bone.

22. The method of claim 13, where the threads are configured as self-tapping and/or self-drilling thread(s).

23. The method of claim 22, where the shank defines a longitudinal, self-tapping notch extending through a portion of the thread(s) from the distal tip toward the proximal head.
